(19) United States
(12) Patent Application Publication
(10) Pub. No.: US 2008/0058775 A1
(43) Pub. Date: Mar. 6, 2008

(54) Ultrasonic debrider probe and method of use

(76) Inventors: Alexander L. Darian, Brightwaters, NY (US); Theodore A.D. Novak, Northport, NY (US); Dan Voic, Cedar Grove, NJ (US)

Correspondence Address: COLEMAN SUDOL SAPONE, P.C., 714 COLORADO AVENUE, BRIDGE PORT, CT, 06605-1601, US

(21) Appl. No.: 11/511,870

(22) Filed: Aug. 29, 2006

(51) Int. Cl. A61B 18/00

(52) U.S. Cl. 606/1

(57) ABSTRACT

An ultrasonic medical probe has an elongate shaft with a head portion having a distal end face oriented at least partially transversely to a longitudinal axis of the shaft. The head portion has a lateral surface extending substantially parallel to the longitudinal axis, the lateral surface being provided with outwardly or radially extending projections. The shaft of the probe is provided with an internal longitudinal channel or bore and at least one ancillary or tributary channel communicating at an inner end with the longitudinal channel or bore and extending to the lateral surface. The ancillary or tributary channel has an outer end disposed in a region about the projections. The projections may be finely configured and distributed so as to form a knurled surface on the head portion.

### BACKGROUND OF THE INVENTION

This invention relates to ultrasonic surgical instruments and associated methods of use. More particularly, this invention relates to high-efficiency medical treatment probes for ultrasonic surgical aspirators. These probes increase the ability to fragment and emulsify hard and soft tissue in a clinical environment while reducing unwanted heat and collateral tissue damage.

Over the past 30 years, several ultrasonic tools have been invented which can be used to ablate or cut tissue in surgery. Such devices are disclosed by Wuchinich et al. in U.S. Pat. No. 4,223,676 and Idemoto et al. in U.S. Pat. No. 5,188,102.

In practice, these surgical devices include a blunt tip hollow probe that vibrates at frequencies between 20 kc and 100 kc, with amplitudes up to 300 microns or more. Such devices ablate tissue by producing cavitation bubbles which implode and disrupt cells, by generating tissue compression and relaxation stresses (sometimes called the jackhammer effect), or by inducing other phenomena such as mechanical shearing and micro streaming of bubbles in the tissue matrix. The effect is that the tissue becomes fragmented and separated. It then becomes emulsified with the irrigant solution. The resulting emulsion is then aspirated from the site. Bulk excision of tissue is possible by applying the energy around and under an unwanted tissue mass to separate it from the surrounding structure. The surgeon can then lift the tissue out using common tools such as forceps.

The hollow or tubular probe is excited by a transducer of either the piezoelectric or magnetostrictive type that transforms an alternating electrical signal within the frequencies indicated above into a longitudinal or transverse vibration. When the probe is attached to the transducer, the two become a single element with series and parallel resonances. The designer will try to tailor the mechanical and electrical characteristics of these elements to provide the proper frequency of operation. Most of the time, the elements will have a long axis that is straight and has the tip truncated in a plane perpendicular to the long axis, as shown in FIG. 1. This is done for simplicity and economic considerations. In almost all applications, whether medical or industrial, such an embodiment is practical and useful. However, in applications where hard tissue such as bone is to be cut, the blunt straight probe has been shown to be less effective. When the tip of the probe is placed against the bone, heating and subsequent charring of the bone tissue takes places. This in turn causes necrosis of the bone which has deleterious effects on healing. Several devices have been invented to cut bone using ultrasound energy. U.S. Pat. Nos. ______ and ______ are examples of devices which may effectively cleave bone for applications such as sinus lifts, laminectomies and chin grafts.

There are applications where cleaving of bone is not required, but instead a sculpting or shaving effect is needed. These indications would include foramanectomies, acoustic neuromas, acromeglias and mandibular shaving. In all of these applications, a grinding action would be needed, not a cutting action. The inventions noted as well as others described in the art are not suited to this requirement.

Therefore, it is desired to provide a probe that can be mated to an ultrasonic surgical aspirator that can sculpt bone without incurring charring of the tissue.

### OBJECTS OF THE INVENTION

An object of the present invention is to provide an improved ultrasonic surgical instrument for use in bone sculpting.

A more particular object of the present invention is to provide such an instrument in the form of a probe that may be used in conjunction with ultrasonic surgical aspirators to sculpt bone.

Another relatively specific object of the present invention is to provide an improved ultrasonic surgical instrument with a form that enhances surgical efficiency and reduces the time required to complete at least some kinds of sculpting procedures.

It is a further object of the present invention to provide such an improved ultrasonic surgical instrument with irrigation and/or suction capability.

It is an additional object of the present invention to provide an improved ultrasonic surgical instrument that may be used in sculpting bone for therapeutic purposes.

It is an additional object of the present invention to provide an improved ultrasonic surgical instrument that may be used in sculpting bone for aesthetic purposes.

An additional object of the present invention is to provide an improved ultrasonic surgical instrument that has liquid directing orifices for greater heat reduction at the operative faces.

These and other objects of the invention will be apparent from the drawings and descriptions herein. Although every object of the invention is attained in at least one embodiment of the invention, there is not necessarily any embodiment which attains all of the objects of the invention.

### SUMMARY OF THE INVENTION

An ultrasonic medical probe in accordance with the present invention comprises an elongate shaft provided with a head portion having a distal end face oriented at least partially transversely to a longitudinal axis of the shaft. The head portion has a lateral surface extending substantially parallel to the longitudinal axis, the lateral surface being provided with at least one outwardly or radially extending projection. The shaft of the probe is provided with an internal longitudinal channel or bore and the probe head is provided with at least one ancillary or tributary channel communicating at an inner end with the longitudinal channel or bore and extending to the lateral surface. Preferably, the ancillary or tributary channel has an outer end disposed in a region about the projection. Also, the projection is preferably one of multiple projections extending from the lateral surface in the region. More preferably, the projections are finely configured and distributed so as to form a knurled surface on the head portion.

The lateral surface may take the form of a cylindrical section. The multiple projections may be pyramidal and disposed in two sets of mutually interleaved rows, the projections in one of the sets of rows being angularly staggered relative to the projections in the other of the sets of rows. Where the rows are each disposed in a respective plane oriented perpendicularly to the longitudinal axis of the probe shaft, the projections in every other row are longitudinally aligned with each other, and the projections in adjacent rows are circumferentially out of alignment with each other.

In an alternative embodiment, the head portion has a plurality of planar lateral faces and the projections are disposed along less than all of the lateral faces. The projections may have a shape taken from the group consisting of pyramids, semi-cylinders, wedges, plates, and flaps or flattened plates.

Where the projection is one of a multiplicity of projections disposed in a region of the lateral surface along one side of the head portion, the ancillary or tributary channel may be one of a plurality of ancillary or tributary channels each communicating at an inner end with the longitudinal channel or bore and extending to the lateral surface.

The ancillary or tributary channel or channels are typically oriented substantially perpendicularly to the longitudinal channel or bore.

Pursuant to another feature of the present invention, the probe further comprises a sheath disposed about the shaft to define therewith an annular channel, the shaft being provided with at least one transverse channel communicating at an inner end with the longitudinal channel or bore and at an outer end with the annular channel. Thus, a portion of an irrigation liquid being delivered to the surgical site for cooling purposes is diverted into the annular channel about the probe shaft, also for cooling purposes. The annular channel is operatively connected to a suction source for aspirating a tissue fragment slurry from the operative site. At times when no slurry is being drawn away through the annular channel, the diverted irrigation liquid still enters the annular channel and cools the outer surface of the probe shaft. Even when slurry is being drawn from the operative site, liquid from the central channel flows together with the slurry through the annular channel, reducing the temperature of the slurry and enhancing the cooling of the outer surface of the probe shaft.

A surgical method in accordance with the present invention utilizes a probe vibratable at at least one ultrasonic frequency, the probe having a distal end face oriented at least partially transversely to a longitudinal axis of the shaft, the probe also having a lateral surface extending substantially parallel to the longitudinal axis, the lateral surface being provided with at least one outwardly or radially extending projection. The method comprises further steps of (a) bringing the lateral surface together with the projection into contact with organic tissues of a patient, (b) during the contacting of the tissues with the lateral surface and the projection, energizing the probe to vibrate the lateral surface and the projection at the ultrasonic frequency, and (c) also during the contacting of the tissues with the lateral surface and the projection, conducting liquid to the lateral surface in a region about the projection via a channel in the probe, the channel extending to the lateral surface.

The bringing of the lateral surface together with the projection into contact with organic tissues of a patient may include inserting a distal end portion of the probe into a fissure or recess in an organ of the patient and moving the probe so that the lateral surface and the projection contact a wall of the fissure or recess. Alternatively or additionally, the bringing the lateral surface together with the projection into contact with organic tissues of a patient may include manipulating the probe so that the lateral surface is oriented substantially parallel to the organic tissues and so that the end face is oriented substantially perpendicularly to the organic tissues immediately prior to an engaging of the organic tissues with the lateral surface and the projection.

Where the probe is provided with a sheath defining an annular channel about the probe, the method further comprises conducting liquid to the annular channel from a longitudinal channel in the probe through a transverse channel or passageway in the probe.

An ultrasonic medical probe comprises, in accordance with a certain embodiment of the present invention, an elongate shaft provided with a head portion having a distal end face oriented at least partially transversely to a longitudinal axis of the shaft and further having a lateral surface extending substantially parallel to the longitudinal axis. The shaft is provided with an internal longitudinal channel or bore, while a sheath being disposed about the shaft to define therewith an annular channel. The probe is provided with at least one transverse channel communicating at an inner end with the longitudinal channel or bore and at an outer end with the annular channel.

A related surgical method comprises bringing the head portion of the probe into contact with organic tissues of a patient, energizing the probe to vibrate the head portion at the ultrasonic frequency during the contacting of the tissues with the head portion, and conducting liquid to the annular channel from the longitudinal channel through the transverse channel during the contacting of the tissues with the head portion.

In a probe in accordance with the present invention, the proximal end of the longitudinal channel or bore in the shaft communicates with a bore in an ultrasonic handpiece using methods well known to the art, such as a male/female thread combination. The probe is shaped such as to provide both a resonant frequency of operation in the range for which the electronic generator was designed and an amplitude of vibration at the distal face which is desired for proper tissue ablation. Such amplitudes have generally been shown to be in the range of 30 to 300 microns. Again, the technique needed for calculating or designing the probe shapes is well known to the art and outside the scope of this disclosure.

Probe ends pursuant to the present invention include features for improving the liquid flow to the probe/tissue interface such as to reduce the bulk temperature rise of the tissue and prevent clogging of the liquid passageway. The projections on the lateral surfaces of the probe heads are energy directors that impart energy from the sides of the probes instead of only at the distal face of the probe. Such energy directors, when contacting skin or tissue, will increase volume of tissue treated per unit time and thereby reduce the operating time of the procedure.

### BRIEF DESCRIPTION OF THE DRAWINGS

FIG. 1 is a cross sectional view of a prior art ultrasonic probe for use with an ultrasonic aspirator.

FIG. 2A is partially a side elevational view and partially a cross-sectional view of an ultrasonic tissue-ablation or debridement probe.

FIG. 2B is a distal end elevational view of the probe of FIG. 2A.

FIG. 2C is partially a top elevational view and partially a cross-sectional view of the probe of FIG. 2A.

FIG. 3A is partially a side elevational view and partially a cross-sectional view of another ultrasonic probe.

FIG. 3B is a distal end elevational view of the probe of FIG. 3A, showing a modification in the form of an elongate groove in a distal end face of the probe head.

FIG. 3C is a view similar to FIG. 3A showing the groove of FIG. 3B.

FIG. 3D is a partial cross-sectional view taken along line III-III in FIG. 3C.

FIG. 4 is partially a side elevational view and partially a cross-sectional view of an ultrasonic probe in accordance with the present invention.

FIG. 4A is partial view, on a larger scale, of a lateral surface of a head of the probe of FIG. 4, taken in region IV-IV of FIG. 4.

FIGS. 4B-4D are side elevational views of the probe head of FIG. 4, showing respective modifications of formations along the lateral surface thereof.

FIG. 4E is a perspective view of the probe head depicted in FIG. 4D.

FIG. 5 is partially a side elevational view and partially a cross-sectional view of yet another ultrasonic probe in accordance with the present invention.

FIG. 6 is a schematic side elevational view of an ultrasonic tissue ablation tool in accordance with the present invention, including a probe head, a sheath and a handpiece, particularly useful in bone sculpting procedures.

FIG. 7 is a partial schematic side elevational view similar to FIG. 6 but on a substantially larger scale, showing the probe head and sheath of FIG. 6.

FIG. 8 is a partial longitudinal cross-sectional view of the probe head and sheath, as well as a probe shaft, shown in FIGS. 6 and 7.

### DETAILED DESCRIPTION

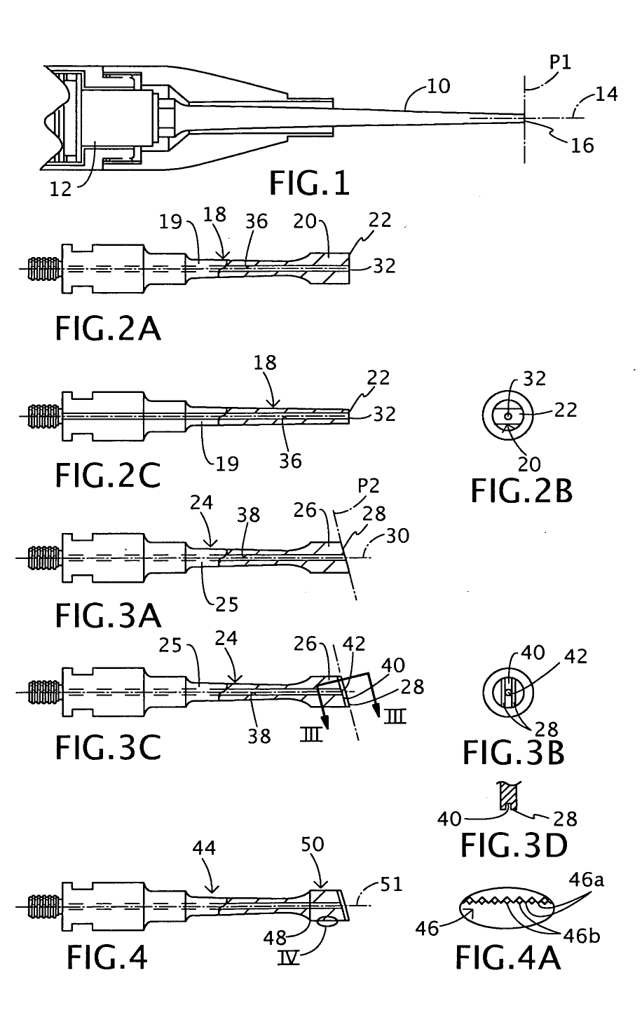

Several probes are disclosed which embody the improvements described herein. FIG. 1 shows a probe 10 which is known to the art and is currently manufactured for use with an ultrasonic aspirator. This probe 10 is basically shaped with an exponential or Gaussian taper. Probe 10 is cannulated and has an integral male thread (not shown) at the proximal end (proximate the operator). This thread communicates with a female threaded bore (not illustrated) in the transducer 12. By tightening the probe 10 onto the transducer 12 and using standard wrenches for final torquing, the transducer and probe essentially become one resonant body. Bores of the probe 10 and transducer 12 communicate with one another. The probe 10 is generally constructed of an acoustically efficient metal or ceramic. Titanium is the most commonly used material, but other material has been employed with success. Material choice does not have a significant impact upon the embodiments of this disclosure.

The distal end of the prior art probe 10 is truncated in a plane P1 perpendicular to the longitudinal axis 14 of the resonant body (probe and transducer). Since the probe 10 is cannulated, a distal end face 16 takes the form of an annular surface with a small cross sectional area. The shape of the probe 10 allows the probe to become a velocity transformer, i.e., the probe will amplify the input vibrations from the transducer 12 by a fixed value, called a gain factor, determined by the geometry of the probe. For example, if the probe 10 had a gain factor of 10, the probe would multiply the input vibration of the transducer, for example 30 microns, to a final amplitude at the distal end of the probe of 300 microns. This phenomenon is well known to the art. By placing the distal end face 16 of probe 10 against organic tissue of a patient, the tissue will be disrupted through cavitation and mechanical effects. By adding saline or water to the tissue-probe interface, cooling of the tissue is achieved and the tissue is emulsified into the liquid and is more easily aspirated either through the center of the probe 10, if the center bore is connected to the aspirator or by separate suction cannulae if the center bore is connected to the irrigant source.

However, the distal end of probe 10 in its conventional configuration is not conducive to ablating large volumes of tissue in short periods of time. By increasing the surface area of distal end face 16, a probe can be constructed which will ablate tissue faster and allow for a shorter operation. This is especially advantageous when debriding wounds such as bedsores, diabetic ulcers, burn wounds, etc.

FIGS. 2A-2C show a probe 18 with a shaft 19 and an enlarged distal head 20. More particularly, probe head 20 may be asymmetrical such that the cross sectional shape is rectangular or oval (see FIG. 2B). This asymmetry allows the probe 18 to maintain a higher gain factor and be more able to be inserted into smaller wounds. The surface area of a distal end face 22 of probe head 20 is greatly increased over the prior art probe (FIG. 1) and will naturally ablate tissue at a higher rate. The shape of the probe head 20 allows access to irregularly shaped wound beds, such as cuts or fissures with slit openings.

Although the probe of FIGS. 2A-2C has been shown to have higher performance over prior art, further improvements may be made. FIG. 3A depicts a probe 24 having a shaft 25 and an asymmetrically enlarged head 26 with a truncated or beveled distal end face 28 located in a plane P2 that is not perpendicular to a longitudinal axis 30 of the probe. This probe 24 has been shown to improve performance in removing the hard eschar buildup of burn wounds, which must be removed in order to expose healthy tissue.

One problem that is encountered in such probe designs, whether the probe head is truncated in a perpendicular plane P1 such as head 20 or in a plane P2 inclined relative to the instrument axis 30 such as probe head 26, is the bore opening 32 or 34 may become blocked with tissue. This blockage prevents aspiration of the emulsified tissue, if the respective bore 36 or 38 is connected to a vacuum source (not shown) or blocks the flow of cooling fluid out of the probe, if the bore is attached to a pressurized liquid source (not shown). Because of the pressure buildup, the liquid has a tendency to jet or stream from the probe tissue interface, causing the irrigant to be sprayed around the room instead of onto the wound bed. Also, if the distal end face of the probe is very large, the liquid may not cover the entire face, even if the opening 32, 34 at the end of the probe is not blocked.

In order to improve the performance of the probe 24 in this regard, a channel, groove, indentation, or notch 40 is provided in the face 28 of the probe, as shown in FIGS. 3B, 3C and 3D. This channel 40 reduces the likelihood of blockage of an output opening 42 of the probe bore 38 by locating this opening or outlet proximally from the distal end face 28 of the probe head 26, while allowing the liquid to fill the channel 40 and cover the remaining distal surface area more fully. Many alternative shapes of channels may be employed in the distal end faces of ultrasonic probes without changing the concepts outlined herein. In the illustrated example, channel or groove 40 extend parallel to or in a length dimension of the end face 28.

When bore 38 is connected to a suction source (not shown), fluid in the channel 40 flows toward the bore 38. When the channel or bore 38 is connected to a source of irrigation liquid (not shown), liquid in the channel 40 flows away from the bore 38.

Regardless of the shape of the distal surface or end faces of the probes as discussed hereinabove, the probes are limited in their ability to ablate tissue by the fact the only area where this ablation can occur is at the distal end face. The sides or lateral surfaces of the probes are generally disposed parallel to the longitudinal axes and parallel to the direction of ultrasonic compression wave transmission. When tissue touches these lateral surfaces, no ablation occurs since the motion is a sliding or rubbing action, which does not transmit sufficient energy into the tissue to cause emulsion or ablation. It is therefore desired to improve ultrasonic tissue ablation probes so that energy may be transmitted from one or more lateral faces or side surfaces of the probe heads so that more tissue may be ablated per unit time.

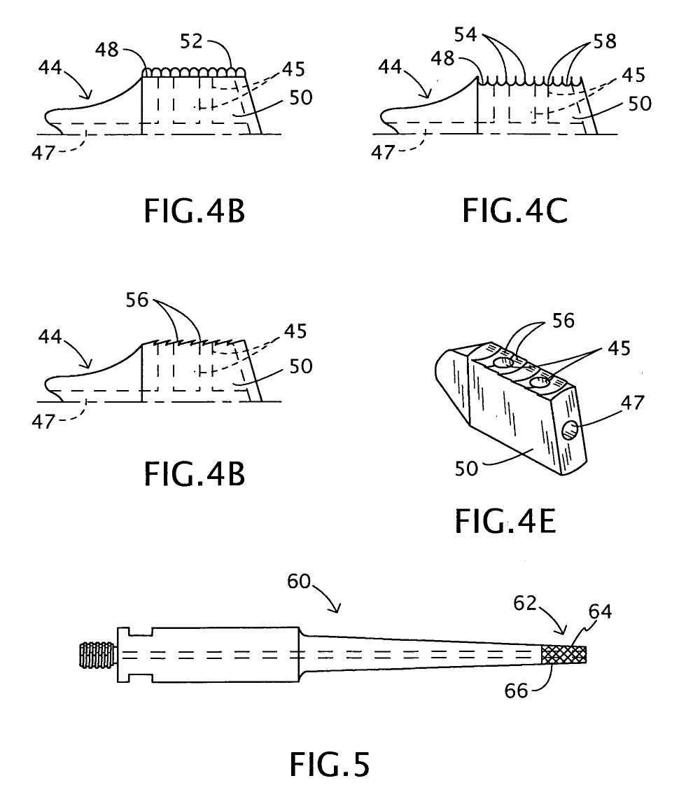

FIGS. 4 and 4A show a probe 44 which is identical to probe 24 of FIGS. 3B-3D with the addition of outwardly or radially extending projections 46 serving as energy guides or directors disposed along at least one lateral or side surface 48 of a probe head 50. Preferably, probe head 50 has a prismatic shape with four planar lateral surfaces or faces 48, projections 46 being disposed only along one or two of the lateral surfaces. As depicted in FIG. 4, energy-directing projections 46 are disposed only along two opposing lateral surfaces 48. Where projections occur along only one or at most two lateral surfaces 48, it is easier for the user to avoid contact with non-target tissues.

Probe head 50 may be integrally formed with a shaft portion 49 of probe 44. Alternatively, probe head 50 may be formed as a separate piece that is firmly attached to shaft 49, e.g., via mating screw threads (not shown) or a force or friction fit. These same alternatives also apply to probe heads 20, 26, 66.

Projections 46 may have a fine geometrical configuration and distribution so as to form the respective lateral surface 48 into a knurled surface as one would find, for example, on a metal file. Or projections 46 may be a series of ridges or knurls on probe head 50. Alternatively, as shown in FIG. 4B, projections or energy directors 46 may be pyramidal sections fashioned from the base metal of the probe 44 that project out in a substantially perpendicular direction from a longitudinal axis 51 of the probe. More specifically, projections or energy directors 46 are a series of parallel ridges or knurls each of triangular cross-section extending transversely to a direction of ultrasonic wave propagation. Projections or energy directors 46 may include a first set of parallel ridges 46a and a second set of ridges 46b that is staggered relative to the first set. Each set of wedge- or triangle-shaped projections or ridges 46a, 46b defines a corresponding set of grooves (not separately designated) each of triangular cross-section extending transversely to a direction of ultrasonic wave propagation. The resulting faceted surfaces of projections or ridges 46a, 46b impart a vector force on the target tissue when the probe 44 vibrates, which will cause cavitation and emulsification of the tissue when it contacts the faceted surfaces.

As further illustrated in FIG. 4B, probe head 50 is optionally provided with one or more transversely oriented tributary channels or bores 45 that communicate on an inner end with a longitudinal central channel or bore 47 and extend to lateral surface 48 for irrigating and cooling energy-directing projections 46.

As illustrated in FIGS. 4B-4E, lateral surface 48 may be provided with energy-directing projections or ridges 52, 54, 56 of different geometrical shapes. Projections or ridges 52 are convex, for instance, semi-cylindrical. Projections or ridges 54 define concave grooves or recesses 58. Projections 56 are flattened plates or flaps that lie against lateral surface 48 in the natural of fish scales. These energy directors or projections 52, 54, 56 allow faster tissue ablation by creating a much larger active surface area at the distal end of the probe 44.

In cases where a probe tip must be smaller than that allowed by the described embodiment, such as when small and/or deep bedsores or wounds must be debrided, the probe tip may be improved to allow faster ablation as well. FIG. 5 shows a probe 60 in the configuration of a tubular end or head 62. Probe 60 is provided circumferentially along a cylindrical lateral or side surface 64 or probe head 62 with a plurality of pyramidal energy-directing projections 66. Projections 66 may be small such as that which occurs in a knurled surface, for example, on a metal file. The energy directors 66 will impart vector forces on the tissue when in contact with the wound bed such that emulsion and ablation will occur around the probe as well as in front of it. Such probes have been shown to increase the speed of ablation and thereby significantly reduce the time of operation. Again, such energy directors may be purely pyramidal, or have concave or convex faces.

All said probes in this embodiment might be designed by those skilled in the art using known tools and techniques.

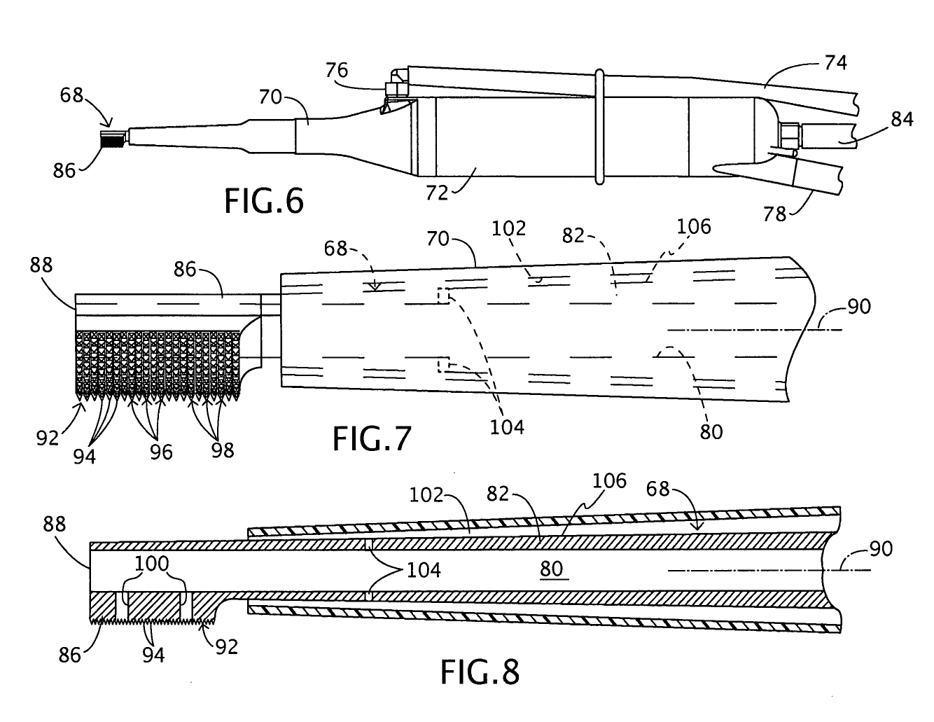

In a method of using the above-described probes for debriding and cleaning wounds, sores and ulcers with ultrasound energy, an operator assembles the ultrasonic surgical aspirator with the probes, connects the central bore to a pressurized liquid source which can be adjusted to provide a controlled flow at the probe tip, turn on the system to provide between 30 and 350 microns of probe tip displacement, and touches the tip and the energy directors to the tissue to be ablated, causing cavitational and mechanical forces to be imparted to said tissue which ablates the tissue, thereby debriding and cleansing the wound bed. Aspiration may be accomplished simultaneously or separately from ultrasonic ablation by connecting a flue or sheath around said probe, as in FIG. 6, that is in turn connected to a vacuum source and then the emulsified tissue is aspirated through this annular space. Conversely, the flue or sheath may be eliminated and the aspirate removed via separate suction cannulae.

A surgical method utilizing probe 24 or 44 or another probe provided in an end face with a channel, groove, indentation, or notch such as channel 40 is operated to vibrate at an ultrasonic frequency. The distal end face 22, 28 of the probe is brought into contact with organic tissues of a patient. The probe is energized to ultrasonically vibrate the end face 22, 28 during the contacting of the tissues with the distal end face, and liquid is channeled between the contacted tissues and longitudinal bore 36, 38, during the contacting of the tissues with the distal end face, via indentation or channel 40.

A surgical method utilizing probe 44 or 60 comprises bringing the lateral surface 48 or 64 together with projections, ridges, or knurls 46, 66 into contact with organic tissues of a patient and, during the contacting of the tissues with the lateral surface and the projections, energizing the probe to vibrate the lateral surface 48, 64 and the projections 46, 66 at a predetermined ultrasonic frequency. This method may include inserting a distal end portion of the probe into a cut, fissure or recess in an organ of the patient and moving the probe so that the lateral surface 48, 64 and the projections 46, 66 contact a wall of the fissure or recess.

Alternatively or additionally, the probe is manipulating so that the lateral surface 48, 64 is oriented substantially parallel to the organic tissues and so that the distal end face is oriented substantially perpendicularly to the organic tissues immediately prior to an engaging of the organic tissues with the lateral surface 48, 64 and the projections 46, 66.

As illustrated in FIG. 6, an ultrasonic medical treatment tool comprises an elongate probe 68, a sheath 70 surrounding a major portion of the probe, and a handpiece 72 connected to proximal ends of the probe and the sheath. Handpiece 72 carries an ultrasonic transducer assembly (not shown) such as that described in U.S. Pat. No. 5,371,429. A suction conduit 74 is connected to a proximal end of sheath 70 via a fitting 76 for extracting liquid containing tissue debris from a surgical site during an ultrasonic bone shaving or sculpting procedure. An irrigation conduit 78 is connected to a proximal end of handpiece 72 for delivering a cooling liquid to the surgical site through a longitudinal channel or bore 80 (FIG. 8) in a shaft 82 of probe 68. An electrical cable 84 is operatively coupled to the proximal end of handpiece 72 for delivering electrical current to power the transducer array.

As shown in FIGS. 6-8, shaft 82 of probe 68 is provided with a head portion 86 having a distal end face 88 oriented transversely or perpendicularly to a longitudinal axis 90 of shaft 82. Head portion 86 has a lateral surface 92 in the form of a cylindrical section (a semi-cylinder) extending substantially parallel to longitudinal axis 90. Lateral surface 92 is formed with a knurled array of pyramidal projections 94 disposed in two circumferentially staggered sets 96 and 98. More specifically, projections 94 are disposed in two sets 96 and 98 of mutually interleaved rows, the projections 94 in row set 96 being angularly staggered relative to the projections in row set 98. Each row 96 and 98 of projections 94 is disposed in a respective plane oriented perpendicularly to longitudinal axis 90 of probe shaft 82. The projections 94 in rows 96 are longitudinally aligned with each other and the projections 94 in rows 98 are longitudinally aligned with each other, while the projections in adjacent rows 96 and 98 are circumferentially out of alignment with each other.

Probe head 86 is provided with at least one and preferably a plurality of ancillary or tributary channels 100 communicating at an inner end with longitudinal channel or bore 80 and extending to lateral surface 92. Ancillary or tributary channels 100 have outer ends disposed in the region of energy-directing projections 94. Ancillary or tributary channels 100 are typically oriented substantially perpendicularly to the longitudinal channel or bore 80 and axis 90.

Sheath 70 and probe shaft 82 together define an annular suction channel or passageway 102. As shown in FIGS. 7 and 8, shaft 82 is provided with at least one and preferably a plurality of transverse channels 104 communicating at inner ends with longitudinal channel or bore 80 and at outer ends with annular channel or passageway 102. Irrigation liquid being delivered to a surgical site via longitudinal channel or bore 80 is partially diverted into annular channel 102, for purposes or enhancing the cooling of shaft 82, particularly an outer surface 106 thereof. Annular channel 102 is operatively connected to a suction source (not shown) via conduit 74 for aspirating a slurry containing tissue fragments or debris from the operative site. At times when no slurry is being drawn away through channel or passageway 102, the irrigation liquid diverted from longitudinal channel 80 still enters the annular channel via transverse channels 104 and cools outer surface 106 of probe shaft 82. Even when slurry is being drawn from the operative site, liquid from central channel 80 flows together with the slurry through annular channel 102, reducing the temperature of the slurry and enhancing the cooling of the outer surface 106 of the probe shaft 82. Transverse channels 102 may be provided in any of the probes disclosed herein or any other ultrasonic probe.

The ultrasonic tool of FIG. 6, particularly including probe 68, is especially efficacious in bone ablation. Probe 68, with head 86, is used to shaving or sculpt bone surfaces. The ultrasonic tool of FIG. 6 is utilized as discussed hereinabove with respect to the probes of FIGS. 1-5, with a modification of the cooling process. During the use of the tool during an ultrasonic bone shaving or sculpting procedure, liquid is conducted to lateral surface 92 via channels 100 to cool the surgical site. In addition, cooling liquid is directed into annular channel or passageway 102 via transverse channels 104.

The bringing of lateral surface 92 together with projections 94 into contact with organic tissues of a patient may include inserting head 86 into a bony fissure or recess in an organ of the patient and manipulating handpiece 72 to move probe 68 so that lateral surface 92 and projections 94 contact a wall of the fissure or recess. Handpiece 72 is further manipulated so that lateral surface 92 is oriented substantially parallel to the bony tissues of the patient and so that end face 88 is oriented substantially perpendicularly to the organic tissues immediately prior to an engaging of the organic tissues with lateral surface 92 and projections 94.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. An ultrasonic medical probe comprising an elongate shaft provided with a head portion, said head portion having a distal end face oriented at least partially transversely to a longitudinal axis of said shaft, said head portion having a lateral surface extending substantially parallel to said longitudinal axis, said lateral surface being provided with at least one outwardly or radially extending projection, said shaft being provided with an internal longitudinal channel or bore, said head portion being provided with at least one ancillary or tributary channel communicating at an inner end with said longitudinal channel or bore and extending to said lateral surface.

2. The probe defined in claim 1 wherein said ancillary or tributary channel has an outer end disposed in a region about said projection.

3. The probe defined in claim 2 wherein said projection is one of a plurality of projections extending from said lateral surface in said region.

4. The probe defined in claim 3 wherein said projections are finely configured and distributed so as to form a knurled surface on said head portion.

5. The probe defined in claim 4 wherein said lateral surface is a cylindrical section.

6. The probe defined in claim 5 wherein said projections are pyramidal and disposed in two sets of mutually interleaved rows, the projections in one of said sets of rows being angularly staggered relative to the projections in the other of said sets of rows.

7. The probe defined in claim 3 wherein said head portion has a plurality of planar lateral faces, said projections being disposed along less than all of said lateral faces.

8. The probe defined in claim 3 wherein said projections have a shape taken from the group consisting of pyramids, semi-cylinders, wedges, plates, and flaps or flattened plates.

9. The probe defined in claim 1 wherein said projection is one of a multiplicity of projections disposed in a region of said lateral surface along one side of said head portion, said ancillary or tributary channel being one of a plurality of ancillary or tributary channels each communicating at an inner end with said longitudinal channel or bore and extending to said lateral surface.

10. The probe defined in claim 1 wherein said ancillary or tributary channel is oriented substantially perpendicularly to said longitudinal channel or bore.

11. The probe defined in claim 1 wherein said probe further comprises a sheath disposed about said shaft to define therewith an annular channel, said probe being provided with at least one transverse channel communicating at an inner end with said longitudinal channel or bore and at an outer end with said annular channel.

12. A surgical method comprising:
providing a probe vibratable at least one ultrasonic frequency, said probe having a distal end face oriented at least partially transversely to a longitudinal axis of said shaft, said probe also having a lateral surface extending substantially parallel to said longitudinal axis, said lateral surface being provided with at least one outwardly or radially extending projection;
bringing said lateral surface together with said projection into contact with organic tissues of a patient;
during the contacting of said tissues with said lateral surface and said projection, energizing said probe to vibrate said lateral surface and said projection at said ultrasonic frequency; and
during the contacting of said tissues with said lateral surface and said projection, conducting liquid to said lateral surface in a region about said projection via a channel in said probe, said channel extending to said lateral surface.

13. The method defined in claim 12 wherein the bringing of said lateral surface together with said projection into contact with organic tissues of a patient includes inserting a distal end portion of said probe into a fissure or recess in an organ of the patient and moving said probe so that said lateral surface and said projection contact a wall of said fissure or recess.

14. The method defined in claim 12 wherein the bringing said lateral surface together with said projection into contact with organic tissues of a patient includes manipulating said probe so that said lateral surface is oriented substantially parallel to said organic tissues and so that said end face is oriented substantially perpendicularly to said organic tissues immediately prior to an engaging of said organic tissues with said lateral surface and said projection.

15. The method defined in claim 12 wherein said probe is provided with a sheath defining an annular channel about said probe, further comprising conducting liquid to said annular channel from a longitudinal channel in said probe, the conducting of liquid including guiding liquid through a transverse channel or passageway in said probe.

16. An ultrasonic medical probe comprising an elongate shaft provided with a head portion, said head portion having a distal end face oriented at least partially transversely to a longitudinal axis of said shaft, said head portion having a lateral surface extending substantially parallel to said longitudinal axis, said shaft being provided with an internal longitudinal channel or bore, a sheath being disposed about said shaft to define therewith an annular channel, said probe being provided with at least one transverse channel communicating at an inner end with said longitudinal channel or bore and at an outer end with said annular channel.

17. A surgical method comprising:
providing a probe vibratable at least one ultrasonic frequency, said probe having a head portion and a longitudinal channel, said probe being provided with a sheath defining an annular channel about said probe, said probe also having at least one transverse channel communicating at an inner end with said longitudinal channel and at an other end with said annular channel;
bringing said head portion into contact with organic tissues of a patient;
during the contacting of said tissues with said head portion, energizing said probe to vibrate said head portion at said ultrasonic frequency; and
during the contacting of said tissues with said head portion, conducting liquid to said annular channel from said longitudinal channel through said transverse channel.
